(19) United States
(12) Patent Application Publication
(10) Pub. No.: US 2019/0004035 A1
(43) Pub. Date: Jan. 3, 2019

(54) MEANS AND METHODS FOR DETERMINATION OF A METABOLIC STATE OF A PLANT

(71) Applicant: BASF PLANT SCIENCE COMPANY GMBH, Ludwigshafen (DE)

(72) Inventors: Elie Fux, Muenchen (DE); Martin Dostler, Henningsdorf (DE); Philipp Ternes, Berlin (DE)

(21) Appl. No.: 16/064,672

(22) PCT Filed: Dec. 21, 2016
(86) PCT No.: PCT/IB2016/001798

§ 371 Date: Jun. 21, 2018

(30) Foreign Application Priority Data
Dec. 21, 2015 (EP) 15201687.9

(51) Int. Cl. G01N 33/50; G01N 1/40

(52) U.S. Cl. CPC G01N 33/5097; G01N 2001/4038; G01N 1/40

(57) ABSTRACT

Provided herein is a method for determining a metabolic state of a plant or part thereof comprising a) rapid evaporating a multitude of metabolites of said plant or part thereof; b) determining the amount of at least one metabolite characteristic of said metabolic state; and c) thereby, determining a metabolic state of a plant thereof. Further provided is a method for in vivo determining a metabolite distribution in a plant or part thereof comprising a) in vivo rapid evaporating at least one metabolite of interest in at least a first and a second location of said plant or part thereof; b) determining the amounts of at least one metabolite at said first and a second location; and, c) thereby, in vivo determining metabolite distribution in a plant or part thereof. Moreover, provided are devices, data collections, and uses relating to the aforesaid methods.

The present invention relates to a method for determining a metabolic state of a plant or part thereof comprising a) rapid evaporating a multitude of metabolites of said plant or part thereof; b) determining the amount of at least one metabolite characteristic of said metabolic state; and c) thereby, determining a metabolic state of a plant thereof. The present invention further relates to a method for in vivo determining metabolite distribution in a plant or part thereof comprising a) in vivo rapid evaporating at least one metabolite of interest in at least a first and a second location of said plant or part thereof; b) determining the amounts of at least one metabolite at said first and a second location, and, c) thereby, in vivo determining metabolite distribution in a plant or part thereof. Moreover, the present invention relates to devices, data collections, and uses relating to the aforesaid methods.

Metabolomics methods have been successfully applied in medical and biologic research, but also in plant research. In higher plants, metabolomics is hampered by an increased complexity of the anatomy of the cell, e.g. by the presence of additional compartments, e.g. plastids.

Complexity of the metabolome of a plant typically is also increased in comparison to, e.g. mammals, since many plants have a pronounced secondary metabolism in addition to the primary metabolism. Accordingly, extracting and analysing single metabolites or groups of metabolites can be challenging.

Accordingly, means and methods were developed enabling simultaneous analysis of a multitude of metabolites, most of which are based on extracting metabolites, ionizing cellular constituents, followed by mass spectrometry (MS) analysis; cf. e.g. WO 2012/068217 A2 for the use of such methods in plant stress analysis. To ease experimentation in the field, portable MS devices were developed (Chen et al. (2014), J AM Soc Mass Spectrom 26:240). More recently, a variety of methods avoiding a dedicated extraction step were devised, in particular methods referred to as “ambient mass spectrometry” methods, generally using direct ionization methods (reviewed in Klampfl & Himmelsbach (2015), Anal Chim Acta 890:44; Takyi-Williams et al. (2015), Bioanalysis 7(15):1901). In plants, e.g. leaf spray ionization coupled to MS was used to analyze specific constituents of the plant (Liu et al. (2011), Anal Chem 83: 7608); the method, however, requires introducing cuts into leaves for analysis, incurring the risk of a defense reaction by the plant, as well as the risk of infection. Moreover, the results are strongly affected by the kind of solvent applied for ionization.

The concept of using high-frequency alternating current to heat tissue has been used in electrosurgical instrumentation for centuries. More recently, methods were developed of aspiring the resultant “surgical smoke” and feeding it into an MS device, termed “rapid evaporation ionization mass spectrometry” (REIMS) (cf. e.g. US 2014/0326865 A1). Using such a device, cancer tissue could be distinguished from healthy tissue during surgery (Balog et al. (2013), Science Transl Med 5(194):194ra93, WO 2014/142927 A1). REIMS was later used to differentiate between different species of mammals in the identification of food fraud and in differentiation between bacterial species (Balog et al. (2010), Anal Chem 82: 7343; Golf et al. (2015), Anal Chem 87: 2527) and for endoscopic tissue identification (Balog et al. (2015), Angew Chemie Int Ed 54: 11059). Moreover, the technology was adapted for liquid phase sample analysis (US 2014/0353488 A1).

However, there is still a need for means and methods for improved metabolomic analysis of plants, in particular avoiding the drawback of the prior art.

Accordingly, the present invention relates to a method for determining a metabolic state of a plant or part thereof comprising

a) rapid evaporating a multitude of metabolites of said plant or part thereof;

b) determining the amount of at least one metabolite characteristic of said metabolic state; and

c) thereby, determining a metabolic state of a plant or part thereof.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention. The term “about” as used herein includes values differing+/−20%, preferably +/−10%, more preferably +/−5%, even more preferably +/−2%, most preferably +/−1% from the value indicated.

The method for determining a metabolic state of the present invention, preferably, is an in vivo method. Moreover, it may comprise steps in addition to those explicitly mentioned above. For example, further steps may relate, e.g., to plant pretreatment for step (a), calculating a value derived from the determined amounts in step b). Moreover, one or more of said steps may be performed by automated equipment.

Preferably, the method comprises the further step of b1) comparing the amount of said at least metabolite to an amount of the same metabolite determined in a plant or part thereof known to be in said metabolic state and/or determined in a plant or part thereof known not to be in said metabolic state, preferably preceding step c). Preferably, said plant known to be in said metabolic state and/or said plant known not to be in said metabolic state are/is (a) plant(s) of the same species as said plant. Preferably, one of said plant known to be in said metabolic state and said plant known not to be in said metabolic state is a control plant, preferably a plant grown under standard conditions. Also preferably at least one of said plant, known to be in said metabolic state and said plant known not to be in said metabolic state is a transgenic plant. Moreover, one or more of said steps may be performed by automated equipment.

As used herein, the term “plant” relates to a whole plant, a plant part, a plant organ, a plant tissue, or a plant cell. Thus, the term includes, preferably, seeds, shoots, stems, leaves, roots (including tubers), and flowers. Preferably, the term relates to a whole plant, more preferably, a whole living plant, most preferably, a whole, living plant in situ. Preferably, the term “plant” relates to a member of the clade Archaeplastida. Plants that are particularly useful in the methods of the invention include all plants which belong to the superfamily Viridiplantae, preferably Tracheophyta, more preferably Spermatophytina, most preferably monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The term “metabolic state”, as used herein, relates to the entirety of metabolic processes occurring in a plant, preferably occurring such that at least one product of such process is detectable in the plant. As is known to the skilled person, the metabolic state of a plant depends on its genetic material, and on a variety of stimuli, which are endogenous or, preferably exogenous. Well-known exogenous stimuli having an impact on the metabolic state of a plant are in particular illumination (including light intensity, duration of illumination, light quality, and the like), nutrient supply, presence or absence of infectious agents, presence or absence of competitor plants, temperature, and the like. As is also known to the skilled person, a stimulus exceeding a certain, typically species- or cultivar-specific, range, represents a stress condition, causing the plant to enter a stress metabolism, wherein the plant adapts its metabolism in an attempt to cope with the stress condition. Preferably, a stress condition is an abiotic stress condition, or a biotic stress condition, i.e., preferably, the metabolic state is a biotic stress metabolic state or an abiotic stress metabolic state. Preferably, the abiotic stress condition is at least one of drought, heat, cold, nitrogen deprivation, phosphorus deprivation, herbicide treatment, fungicide treatment, and insecticide treatment. Preferably, the biotic stress condition is at least one of fungal infection, bacterial infection, viral infection, and nematode infection. In a preferred embodiment, the biotic stress condition is caused by at least one herbivore parasite and/or arthropode infestation, in particular an insect pest or an arachnid pest.

Preferably, the abiotic stress metabolic state is at least one of drought metabolism, heat metabolism, cold metabolism, nitrogen deprivation metabolism, phosphorus deprivation metabolism, photosynthesis metabolism, herbicide treatment metabolism, fungicide treatment metabolism, and insecticide treatment metabolism.

Also preferably, the biotic stress metabolic state is at least one of metabolism in the presence of fungal infection, preferably in the presence of a particular fungal development stage, metabolism in the presence of bacterial infection, metabolism in the presence of viral infection, and metabolism in the presence of nematode infection. Preferably, in case the stress condition is a biotic stress condition, the term “metabolic state” includes the processes occurring in the plant as a reaction to the infection with the infectious agent, e.g. defense reactions. Also preferably, the term further includes the processes occurring in or caused to occur by the infectious agent itself, in as far as their products are detectable in the plant. Thus, preferably, the method of the present invention further comprises (a) identifying an infectious agent by (aa) detecting at least one metabolite produced by said infectious agent and/or (bb) by detecting at least one metabolite produced by said plant or part thereof in response to said infectious agent; and/or (b) identifying the development stage of an infectious agent by (aa) detecting at least one metabolite specifically produced by said infectious agent in said development stage and/or (bb) by detecting at least one metabolite specifically produced by said plant or part thereof in response to said infectious agent in said development stage. Thus, preferably, the present invention also relates to a method of identifying an infectious agent and/or identifying a development stage of an infectious agent.

Accordingly, the term “determining a metabolic state” relates to determining whether a metabolic state adapting the plant to a specific stimulus, preferably one or more stress condition(s), was triggered in the plant, or not. Preferably, determining a metabolic state relates to determining whether at least one metabolic adaptation was triggered in a plant in response to a stimulus, preferably a stress condition as specified elsewhere herein. Preferably, said at least one metabolic adaptation is an adaptation specific for said stress condition or stress conditions. More preferably, said at least one metabolic adaptation is an adaptation specific for a specific stress condition, preferably as specified above.

According to the invention, preferably, the metabolic state of a whole plant is established; e.g. it is established, whether the plant is under nitrogen deprivation stress, under heat stress, or the like. As will be appreciated by the skilled person, the metabolic state may also be specific for a plant part; e.g. a leaf infected locally by an infectious agent may have a metabolic state at the site of infection and/or in the infected leaf different from the metabolic state of the residual plant parts. Preferably, in determining a metabolic state of a plant or part thereof, the plant part under investigation is not removed from the plant. Thus, the method determining a metabolic state of a plant or part thereof, preferably, is an in vivo method. Preferably, the part of the plant on which rapid evaporation is performed is not removed from the plant. Preferably, the method is an in situ method, i.e. a method wherein determining is performed on a plant without removing said plant from the soil.

Preferably, determining a metabolic state comprises determining a value of at least one parameter of at least one metabolite or multitude of metabolites characteristic of a metabolic state of a plant. As will be understood by the skilled person, in principle, the identity of the metabolite or multitude of metabolites causing the parameter to have the value measured need not be known. Thus, preferably, said at least one parameter characteristic of at least one metabolite or multitude of metabolites characteristic of a metabolic state of a plant is a peak or a pattern in a chromatogram, preferably, is a peak or a pattern in a mass spectrum. As will also be understood by the skilled person, in cases where the presence or absence of a peak or pattern is indicative of a metabolic state, comparison to a reference plant may not be required and, preferably, as noted above, the identity of the metabolite or multitude of metabolites causing the presence or absence of a peak or pattern need not be known.

In a preferred embodiment, values of parameters of a multitude of metabolites are determined. Preferably, outlier values are excluded from the values used in further analysis. Preferably, the values determined are normalized. More preferably, in particular in case the values are determined by mass spectrometry, the values (peak intensities) determined are normalized by a term calculated from the values themselves, e.g. they are normalized by the sum of the values of at least one, preferably more than one, most preferably all parameters determined in each sample or by the median of the values of at least one, preferably more than one, most preferably all parameters determined in each sample. Preferably, the normalized values are further corrected for the influence of one or more confounding factors. More preferably, in particular in case the values are determined by mass spectrometry, one or more of said confounding factors can be described by one or more terms calculated from the values (peak intensities) themselves, before or after normalization. In a preferred embodiment, one of said confounding factors correlates with the sum of the values of at least one, preferably more than one, most preferably all parameters determined in each sample before normalization. In an equally preferred embodiment, one of said confounding factors correlates with the median of the values of at least one, preferably more than one, most preferably all parameters determined in each sample before normalization. In yet another preferred embodiment, one of said confounding factors correlates with the weighted sum of the values of at least one, preferably more than one, most preferably all parameters determined in each sample, preferably after normalization, in which the weight of each parameter is proportional to the relative standard deviation of the corresponding values in all samples. In an even more preferred embodiment, one of said confounding factors correlates with the sum of the values of at least one, preferably more than one, most preferably all parameters determined in each sample before normalization, and another one correlates with the weighted sum of the values of at least one, preferably more than one, most preferably all parameters determined in each sample, calculated as described. In an equally preferred embodiment, one of said confounding factors correlates with the median of the values of all parameters determined in each sample before normalization, and another one correlates with the weighted sum of the values of all parameters determined in each sample, calculated as described. Preferably, the normalized values are analyzed by analysis of variance (ANOVA), more preferably using a mixed effects model, in an embodiment as described herein in the Examples. This embodiment also comprises the use of ANOVA for the correction for confounding factors. As is understood by the skilled person, other ANOVA models may be used, e.g., fixed-effect models, mixed-effect models, or hierarchical models. Further preferred methods for performing principal component analysis (PCA) and/or ANOVA are described herein in the Examples, in particular Example 5.

Preferably, determining a metabolic state is determining the abundance of at least one metabolite of a plant, wherein said metabolite is known to correlate with said metabolic state. Preferably, the presence or absence of said metabolite is indicative of said metabolic state; as will be understood by the skilled person, no direct comparison to a reference will be necessary in such case. Also preferably, the abundance of said metabolite, e.g., preferably, its relative or absolute concentration in a plant or plant part, is indicative of said metabolic state. Preferably, the metabolite is a metabolite as specified elsewhere herein.

The term “evaporating” as used herein, relates to heating a portion of the plant or plant part to produce a vapor comprising metabolites comprised in said portion of the plant or plant part. Preferably, evaporating comprises inducing a heating of said portion of said plant or plant part to a temperature of at least 250° C., more preferably at least 300° C., even more preferably at least 350° C., most preferably at least 400° C. “Rapid evaporation” as used herein, relates to evaporating within a short time. Preferably, rapid evaporating comprises inducing the aforementioned temperatures within at most 10 s, more preferably within 5 s, even more preferably within 2 s, most preferably within 1 s after start of the heating process. Preferably, rapid evaporation is performed on a small area on the intact plant or plant part, preferably an area with a size of at most 1 cm², preferably at most 0.5 cm², more preferably at most 1 mm². Preferably, rapid evaporation is induced by applying a laser pulse to the plant tissue; by applying an electrical heating device, preferably a heating wire or coil, e.g. a nickel-chrome coil; or by applying high-frequency alternating current.

More preferably, rapid evaporation is induced by applying a high-frequency alternating current. Preferably, the high-frequency alternating current has a peak-to-peak voltage of from 400 V to 10,000, more preferably of from 500 V to 6,000 V, most preferably of from 1,000 to 4,000 V. Also preferably, the high-frequency alternating current has a frequency of from 5 kHz to 5 MHz, more preferably of from 10 kHz to 2500 kHz, even more preferably of from 1000 kHz to 2300 kHz or of from 25 kHz to 550 kHz. Most preferably, the high-frequency alternating current has a frequency of 1000 kHz to 2300 kHz. Preferably, the high-frequency alternating current is applied to the plant or part thereof by means of two electrodes of about equal size, preferably the size of the area of rapid evaporation. More preferably, the high-frequency alternating current is applied to the plant or part thereof by means of electrosurgical equipment, most preferably electrosurgical forceps or equivalent means.

Thus, in a preferred embodiment, the high-frequency alternating current is applied to the plant or part thereof by means of a bipolar forceps, i.e., preferably, a forceps of which one tip is an electrode, and the other tip is a counter electrode. Bipolar forceps are known in the art e.g. from electrosurgery and are available with a variety of electrode areas.

In a further preferred embodiment, the high-frequency alternating current is applied to the plant or part thereof by means of a unipolar electrode and a dissipation means. As will be understood by the skilled person, the degree of heating at the electrode and the counter-electrode is governed by the geometry of the electrode and the counter electrode, as well as by the area ratio between the electrode and the counter electrode. Preferably, the area ratio of electrode/counter electrode (dissipation electrode) is at least 100, more preferably at least 1000, most preferably at least 10000. Preferably, in such case, the unipolar electrode comprises a pointed tip, preferably concentrating current in a small area, preferably a small area as specified herein above. Appropriate devices are known in the art and include electrosurgical knives or unipolar forceps, i.e., preferably, forceps of which one or both tips form the electrode. The counter electrode, preferably is an electrode providing a large area of contact to the plant, preferably at least 1 cm², more preferably at least 2 cm², even more preferably at least 10 cm². The counter electrode, preferably, is made of a conductive material. Preferably, the counter electrode comprises a conductive mat brought into contact with the plant or part thereof. More preferably, said mat was wetted before contacting with said plant or plant part. More preferably, the counter electrode comprises an adhesive patch, in an embodiment consists of an adhesive patch, which is, preferably, attached to the plant. As will be understood by the skilled person, the counter electrode may, in principle, be contacted to any part of the plant or part thereof, provided that a conductive connection exists between the electrode and the counter electrode. Preferably, the counter electrode is contacted to the plant or plant part in close proximity to the electrode, e.g. preferably, on the same organ of the plant, e.g. the same leaf; or, more preferably, the counter electrode is brought into contact with the plant or part thereof such that the distance between the electrode and the counter electrode is as short as possible, e.g. preferably, the electrode and the counter electrode are placed on opposing sides of the same leaf.

The term “multitude” is understood by the skilled person. Preferably, the term relates to at least 5, more preferably at least 25, even more at least 50, most preferably at least 100.

The term “metabolite”, as used herein, relates to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as plants. Preferably, a metabolite has a molecular weight of from 25 Da (Dalton) to 300,000 Da, more preferably of from 30 Da to 30,000 Da, most preferably of from 50 Da to 1500 Da. Preferably a metabolite has a molecular weight of less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, or less than 100 Da. Preferably, a metabolite has, however, a molecular weight of at least 50 Da.

Preferably, the metabolite is a biological macromolecule, e.g. preferably, DNA, RNA, protein, or a fragment thereof, more preferably a fragment produced by rapid evaporation of plant tissue. More preferably, in case a plurality of metabolites is envisaged, said plurality of metabolites representing a metabolome, i.e. the collection of metabolites being comprised by an organism, an organ, a tissue, a body fluid or a cell at a specific time and under specific conditions.

More preferably, the metabolite in accordance with the present invention is a small molecule compound, such as a substrate for an enzyme of a metabolic pathway, an intermediate of such a pathway or a product obtained by a metabolic pathway. Metabolic pathways are well known in the art and may vary between species. Preferably, said pathways include at least citric acid cycle, respiratory chain, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including e.g. flavonoids and isoflavonoids), isoprenoids (including eg. terpenes, sterols, steroids, carotenoids, xanthophylls), carbohydrates, phenylpropanoids and derivatives, alcaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal cellular function, organ function or animal growth, development or health. Moreover, small molecule metabolites further comprise secondary metabolites having essential ecological function, e.g. metabolites which allow an organism to adapt to its environment. Furthermore, metabolites are not limited to said primary and secondary metabolites and further encompass artificial small molecule compounds. Said artificial small molecule compounds are derived from exogenously provided small molecules which are administered or taken up by an organism but are not primary or secondary metabolites as defined above, including, preferably, herbicides, fungicides, and insecticides. Moreover, artificial small molecule compounds may be metabolic products of compounds taken up, and preferably metabolized, by metabolic pathways of the plant. Moreover, small molecule compounds preferably include compounds produced by organisms living in, on or in close vicinity to the plant, more prefer ably by infectious agent as specified elsewhere herein.

According to the method of the present invention, at least one metabolite characteristic of a metabolic state is determined. Preferably, this is achieved by selecting detection parameters such that at least one metabolite known to be characteristic of a metabolic state is detected. Also preferably, a multitude of metabolites is detected in a plant known to be in said metabolic state (e.g. a positive control plant) and in a plant known not to be in said state (e.g. a negative control plant); when the detected multitude of metabolites is compared, metabolites and/or patterns corresponding to the plant known to be in said metabolic state, but not in the plant known not to be in in said metabolic state, i.e. characteristic of a metabolic state, can be identified. As will be understood by the skilled person, detection of a metabolite by MS preferably includes detection of the metabolite itself, of one or more fragments thereof and/or of adducts of said metabolite or fragment. Moreover, preferably, depending on the chemical nature of the metabolite, an ionized, more preferably protonated, form of said metabolite, fragment, and/or adduct, is detected in MS.

The term “determining the amount”, in particular of a metabolite, as used herein, refers to determining at least one characteristic feature of a metabolite to be determined in a sample. Characteristic features in accordance with the present invention are features which characterize the physical and/or chemical properties including biochemical properties of a metabolite. Such properties include, e.g., molecular weight, elution time in liquid chromatography or in gas chromatography, fractionation pattern, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a metabolite by standard operations, e.g., mathematical calculations such as multiplication, division or logarithmic calculus. Most preferably, the at least one characteristic feature allows the determination and/or chemical identification of the said at least one metabolite and its amount. Accordingly, the characteristic value, preferably, also comprises information relating to the abundance of the metabolite from which the characteristic value is derived. For example, a characteristic value of a metabolite may be a peak in a mass spectrum. Such a peak contains characteristic information of the metabolite, i.e. the m/z information, as well as an intensity value being related to the abundance of the said metabolite (i.e. its amount) in the sample.

A metabolite may be, preferably, determined in accordance with the present invention qualitatively, e.g. detectable or not detectable; semiquantitatively, e.g. abundant, scarce; or, preferably, quantitatively, e.g., preferably, as a relative or absolute concentration or proportion, e.g. of dry mass. For semi-quantitative determination, preferably, the relative amount of the metabolite is determined based on the value determined for the characteristic feature(s) referred to herein above. The relative amount may be determined in a case were the precise amount of a metabolite can or shall not be determined. In said case, it can be determined whether the amount in which the metabolite is present, is increased or diminished with respect to a second sample comprising said metabolite in a second amount; or it can be determined whether the amount in which the metabolite is present, is increased or diminished with respect to an internal control analyte. A standard compound, preferably, is provided by infusing or otherwise applying a marker compound to the plant, preferably to the area of analysis. Preferably, said standard compound is a compound not naturally present in the plant. More preferably, the metabolite, in particular the diagnostic metabolite, is determined quantitatively, i.e. preferably, determining is measuring an absolute amount or a concentration of a metabolite.

Preferably, the determination of the amount of a metabolite as referred to herein is achieved by an optional compound separation step and a mass spectrometry step. Thus, determining as used in the method of the present invention, preferably, comprises performing direct infusion mass spectrometry; also preferably, determining further includes using a compound separation step prior to the analysis step. Preferably, said compound separation step yields a time resolved separation of the metabolites, in particular of the diagnostic metabolites, comprised by the sample. A preferred technique for separation to be used in accordance with the present invention therefore is ion mobility. Moreover, determination via ion mobility, either as sole separation method or, preferably in combination with MS, more preferably MS/MS, most preferably of one of the combinations specified herein below, is envisaged. These techniques are well known in the art and can be applied by the person skilled in the art without further ado.

Preferably, mass spectrometry (MS) is used for detecting metabolites. Mass spectrometry methods as used herein encompasses all techniques which allow for the determination of the molecular weight (i.e. the mass) or a mass variable corresponding to a compound, i.e. a metabolite, to be determined in accordance with the present invention. Preferably, mass spectrometry as used herein relates to sector MS, Time of flight (TOF) MS, Quadrupole mass filter MS, Ion Trap MS (including, preferably, 3D quadrupole ion trap MS, cylindrical ion trap MS, linear quadrupole ion trap MS, and Orbitrap MS), and/or Fourier transform ion cyclotron resonance MS (FT-ICR-MS). Preferably, mass spectrometry, as used herein, relates to any stage of sequentially coupled mass spectrometry, such as MS-MS or MS-MS-MS, or any combined approaches using the aforementioned techniques. More preferably, TOF-MS and/or quadrupole MS (Q-MS) is used. Most preferably, a combination of TOF-MS and Q-MS is used (Q-TOF-MS). How to apply these techniques is well known to the person skilled in the art. Moreover, suitable devices are commercially available.

For mass spectrometry, the metabolites are ionized by rapid evaporation in order to generate charged molecules or molecule fragments. Afterwards, the mass-to-charge of the ionized analytes, in particular of the ionized metabolites, or fragments thereof is measured. Ionization of the metabolites can be carried out by any method deemed appropriate, as described elsewhere herein.

As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado.

Preferably, steps a) and b) of the method for determining a metabolic state of a plant, i.e., preferably, rapid evaporating a multitude of metabolites of a plant or part thereof and determining the amount of at least one metabolite characteristic of said metabolic state are performed by rapid evaporative ionization mass spectrometry (REIMS).

The method of the present invention shall be, preferably, assisted by automation. For example, data processing and comparison is, preferably, assisted by suitable computer programs and databases. Automation as described herein before allows using the method of the present invention in high-throughput approaches.

Advantageously, it was found in the research underlying the present invention that rapid evaporation can be used to ionize metabolites from small areas from living plants and that the small changes induced by stress factors can be successfully identified. The method of the present invention does not require cutting or otherwise interfering with the structure of plant tissue before ionization, avoiding the risk of inducing unwanted stress reactions to the cutting itself, and avoiding creating open wounds on the plant, bearing the risk of infection.

The definitions made above apply mutatis mutandis to the following. Additional definitions and explanations made further below also apply for all embodiments described in this specification mutatis mutandis.

The present invention also relates to a method for in vivo determining metabolite distribution in a plant or part thereof comprising

a) in vivo rapid evaporating at least one metabolite of interest in at least a first and a second location of said plant or part thereof
b) determining the amounts of at least one metabolite at said first and a second location, and,
c) thereby, in vivo determining metabolite distribution in a plant or part thereof.

The method for in vivo determining metabolite distribution of the present invention is an in vivo method. Thus, preferably, the part or parts of the plant on which rapid evaporation is performed is not removed from the plant. Also, one or more of said steps may be performed by automated equipment. Moreover, the method may comprise steps in addition to those explicitly mentioned above. E.g., preferably, the method further comprises the further step of comparing the amounts of at least one metabolite of interest in said at least first and second location, and/or comparing the metabolite distribution determined to the metabolite distribution in a second plant, preferably a control plant.

As will be understood by the skilled person, the method for in vivo determining metabolite distribution may be a static method determining the distribution of one or more metabolites of interest in the plant. However, the method for in vivo determining metabolite distribution may also be used as a dynamic method determining the distribution of one or more metabolites of interest in the plant over time, thus establishing fluxes, e.g. between tissues and/or organs of the plant.

The terms “first location” and “second location” of a plant or part thereof are understood by the skilled person. Preferably, the terms relate to, preferably non-identical, regions of a plant body.

Preferably, the regions have the size indicated for the area of rapid evaporation elsewhere herein. As will be understood by the skilled person, the positioning of the first and second location on the plant will depend on the question to be answered.

Further, the present invention relates to a device comprising

i) a means for in vivo rapid evaporating at least one metabolite of a plant or part thereof

ii) an analysis unit comprising means for determining the value of at least one parameter characteristic of said at least one metabolite

iii) an evaluation unit comprising a data storage unit and means for comparing the value of said at least one parameter to said reference amount.

A “device”, as the term is used herein, shall comprise at least the aforementioned units. The units of the device are operatively linked to each other. How to link the means in an operating manner will depend on the type of units included into the device. For example, where the analysis unit allows for automatic qualitative or quantitative determination of the metabolite, the data obtained by said automatically operating analyzing unit can be processed by, e.g., a computer program in order to facilitate the assessment in the evaluation unit. Preferably, the units are comprised by a single device in such a case. Preferably, the device includes an analyzing unit for the metabolite and a computer or data processing device as an evaluation unit for processing the resulting data for the assessment and for establishing the output information. Preferably, the analysis unit comprises at least one detector for at least one metabolite according to the present invention. Preferably, in case the device is a device for determining a metabolic state of a plant, the evaluation unit comprises a data storage unit, wherein said data storage unit comprises at least one reference amount for a metabolite, preferably obtained from a plant known to be in specific metabolic state and/or a plant known not to be in said metabolic state. More preferably, the device comprises a library, preferably a spectral library, of reference mass spectra, preferably obtained from a plant known to be in specific metabolic state and/or a plant known not to be in said metabolic state. Also preferably, in case the device is a device for in vivo determining metabolite distribution in a plant or part thereof, the evaluation unit comprises a data storage unit, wherein said data storage unit comprises at least one value reference amount for a metabolite, preferably obtained from a pre-defined location of a pre-defined plant.

Preferred devices are those which can be applied without the particular knowledge of a specialized clinician, e.g., electronic devices which merely require contacting the means for in vivo rapid evaporating with a plant or part thereof. The output information of the device, preferably, is a value or display which allows drawing conclusions on the metabolic state of a plant and/or on metabolite distribution in a plant or part thereof. Preferably, the device is a mobile device, i.e. a device which can be transported to a new location essentially without dismantling. Preferably, the device further comprises a plant or part thereof known to be in a specific metabolic state and/or a plant or part thereof known not to be in said metabolic state.

The present invention also relates to a collection, preferably a database comprising reference values obtained from a plant known to be in specific metabolic state and/or from a plant known not to be in said metabolic state obtained by

a) rapid evaporating at least one metabolite of said plant or part thereof, and

b) determining the amount of at least one metabolite characteristic of said metabolic state.

Further, the present invention relates to a data carrier comprising the data collection, the database, or the data of the data collection of the present invention.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection, preferably on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets, preferably being indicative for a specific metabolic state as set forth above (e.g. a query search). Thus, if an identical or similar data set can be identified in the data collection, the test data set will be associated with the presence of said metabolic state, or not. Consequently, the information obtained from the data collection can be used, e.g., as a reference for the methods of the present invention described above.

The term “data storage medium” as used herein encompasses data storage media which are based on single physical entities such as a CD, a CD-ROM, a hard disk, optical storage media, flash memory, and the like. Moreover, the term further includes data storage media consisting of physically separated entities which are operatively linked to each other in a manner as to provide the aforementioned data collection, preferably, in a suitable way for a query search.

The present invention further relates to a use of a device according to the present invention, for determining a metabolic state of a plant, preferably according to the method of the present invention, and/or for in vivo determining metabolite distribution in a plant or part thereof, preferably according to the method of the present invention.

The invention further discloses and proposes a computer program including computer-executable instructions for performing the method according to the present invention or parts thereof in one or more of the embodiments enclosed herein when the program is executed on a computer or computer network. Specifically, the computer program may be stored on a computer-readable data carrier. Thus, specifically, one, or more than one of the method steps may be performed by using a computer or a computer network, preferably by using a computer program.

The invention further discloses and proposes a computer program product having program code means, in order to perform the method according to the present invention or parts thereof in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Moreover, the invention proposes and discloses a modulated data signal which contains instructions readable by a computer system or computer network, for performing the method according to one or more of the embodiments disclosed herein.

In view of the above, the following embodiments are preferred:

### Embodiment 1

A method for determining a metabolic state of a plant or part thereof comprising

a) rapid evaporating a multitude of metabolites of said plant or part thereof;

b) determining the amount of at least one metabolite characteristic of said metabolic state; and

c) thereby, determining a metabolic state of a plant or part thereof.

### Embodiment 2

The method of embodiment 1, wherein the part of the plant on which rapid evaporation is performed is not removed from the plant.

### Embodiment 3

The method of embodiment 1 or 2, wherein said method is an in vivo method.

### Embodiment 4

The method of any one of embodiments 1 to 3, wherein the amounts of a multitude of metabolites is determined.

### Embodiment 5

The method of any one of embodiments 1 to 4, wherein said rapid evaporating is performed on a small area on the intact plant or plant part.

### Embodiment 6

The method of embodiment 5, wherein said small area is an area with a size of at most 1 cm², preferably at most 0.5 cm², more preferably at most 1 mm².

### Embodiment 7

The method of any one of embodiments 1 to 6, wherein rapid evaporation is induced by applying a laser pulse to said plant or part thereof; by applying heat from an electrical heating device, preferably a heating wire or coil, to said plant or part thereof; or by applying high-frequency alternating current to said plant or part thereof.

### Embodiment 8

The method of any one of embodiments 1 to 7, wherein rapid evaporation is induced by applying high-frequency alternating current to said plant or part thereof.

### Embodiment 9

The method of embodiment 8, wherein said high-frequency alternating current is applied to said plant or part thereof by means of two electrodes of about equal size, preferably the size of the area of rapid evaporation.

### Embodiment 10

The method of any one of embodiments 7 to 9, wherein the high-frequency alternating current is applied to said plant or part thereof by means of electrosurgical equipment, preferably electrosurgical forceps or equivalent means.

### Embodiment 11

The method of any one of embodiments 1 to 10, wherein said at least one parameter characteristic of said at least one metabolite is determined by mass spectrometry.

### Embodiment 12

The method of any one of embodiments 1 to 11, wherein said steps a) and b) are performed by rapid evaporative ionization mass spectrometry (REIMS).

### Embodiment 13

The method of any one of embodiments 1 to 12, wherein said metabolic state is a biotic stress metabolic state or an abiotic stress metabolic state.

### Embodiment 14

The method of embodiment 13, wherein said abiotic stress metabolic state is

(i) drought metabolism,

(ii) heat metabolism,

(iii) cold metabolism,

(iv) nitrogen deprivation metabolism,

(v) phosphorus deprivation metabolism,

(vi) photosynthesis metabolism

(vii) herbicide treatment metabolism,

(viii) fungicide treatment metabolism,

(ix) insecticide treatment metabolism, or

(x) an arbitrary combination of at least two of (i) to (ix).

### Embodiment 15

The method of embodiment 13, wherein said biotic stress metabolic state is

(i) metabolism in the presence of fungal infection, preferably in the presence of a particular fungal development stage,

(ii) metabolism in the presence of bacterial infection,

(iii) metabolism in the presence of viral infection,

(iv) metabolism in the presence of nematode infection; or

(v) an arbitrary combination of at least two of (i) to (iv).

### Embodiment 16

The method of any one of embodiments 1 to 15, wherein said plant is a plant

(i) grown under stress conditions and/or

(ii) infected by an infectious agent.

### Embodiment 17

The method of embodiment 16, wherein said stress conditions are

(i) drought,

(ii) heat,

(iii) cold,

(iv) nitrogen deprivation,

(v) phosphorus deprivation,

(vi) light deprivation,

(vii) overexposure to light,

(viii) herbicide treatment,

(ix) fungicide treatment,

(x) insecticide treatment, or

(xi) an arbitrary combination of at least two of (i) to (x).

### Embodiment 18

The method of any one of embodiments 1 to 17, wherein said method comprises

(a) identifying an infectious agent by (aa) detecting at least one metabolite produced by said infectious agent and/or (bb) by detecting least one metabolite produced by said plant or part thereof in response to said infectious agent; and/or (b) identifying the development stage of an infectious agent by (aa) detecting at least one metabolite specifically produced by said infectious agent in said development stage and/or (bb) by detecting at least one metabolite specifically produced by said plant or part thereof in response to said infectious agent in said development stage.

### Embodiment 19

The method of any one of embodiments 1 to 18, further comprising the step of b1) comparing the amount of said at least metabolite to an amount of the same metabolite determined in a plant or part thereof known to be in said metabolic state and/or determined in a plant or part thereof known not to be in said metabolic state, preferably preceding step c).

### Embodiment 20

The method of embodiment 19, wherein said plant known to be in said metabolic state and/or said plant known not to be in said metabolic state is a plant of the same species as said plant.

### Embodiment 21

The method of embodiment 19 or 20, wherein one of said plant known to be in said metabolic state and said plant known not to be in said metabolic state is a control plant, preferably grown under standard conditions for said plant.

### Embodiment 22

The method of any one of embodiments 19 to 21, wherein at least one of said plant, said plant known to be in said metabolic state and said plant known not to be in said metabolic state is a transgenic plant.

### Embodiment 23

A method for in vivo determining metabolite distribution in a plant or part thereof comprising

a) in vivo rapid evaporating at least one metabolite of interest in at least a first and a second location of said plant or part thereof

b) determining the amounts of at least one metabolite at said first and a second location, and,

c) thereby, in vivo determining metabolite distribution in a plant or part thereof.

### Embodiment 24

The method of embodiment 23, wherein said first and second location are located in different organs of said plant.

### Embodiment 25

The method of embodiment 23 or 24, further comprising comparing the amounts of said at least one parameter characteristic of said at least one metabolite of interest in said at least first and second location.

### Embodiment 26

The method of any one of embodiments 23 to 25, wherein a multitude of metabolites of interest is evaporated.

### Embodiment 27

The method of any one of embodiments 23 to 26, wherein said rapid evaporating in said at least first and second location is performed on small areas on the intact plant or plant part.

### Embodiment 28

The method of embodiments 23 to 27 wherein said first and second location are areas with a size of at most 1 cm2, preferably at most 0.5 cm2, more preferably at most 10 mm2.

### Embodiment 29

The method of any one of embodiments 23 to 28, wherein rapid evaporation is induced by applying a laser pulse to said plant or part thereof; by applying heat from an electrical heating device, preferably a heating wire or coil, to said plant or part thereof; or by applying high-frequency alternating current to said plant or part thereof.

### Embodiment 30

The method of embodiment 29, wherein rapid evaporation is induced by applying high-frequency alternating current to said plant or part thereof.

### Embodiment 31

The method of embodiment 29 or 30, wherein said high-frequency alternating current is applied to said plant or part thereof by means of two electrodes of about equal size, preferably the size of the area of rapid evaporation.

### Embodiment 32

The method of any one of embodiments 29 to 31, wherein the high-frequency alternating current is applied to said plant or part thereof by means of electrosurgical equipment, preferably electrosurgical forceps or equivalent means.

### Embodiment 33

The method of any one of embodiments 23 to 32, wherein the part or parts of the plant on which rapid evaporation is performed is or are not removed from the plant.

### Embodiment 34

The method of any one of embodiments 23 to 34, further comprising the step of comparing the metabolite distribution determined in step c) to the metabolite distribution in a second plant, preferably a control plant.

### Embodiment 35

The method of any one of embodiments 23 to 34, wherein said second plant is a plant of the same species as said plant.

### Embodiment 36

The method of any one of embodiments 23 to 35, wherein said at least one parameter characteristic of said at least one metabolite is determined by mass spectrometry.

### Embodiment 37

The method of any one of embodiments 23 to 36, wherein said steps a) and b) are performed by rapid evaporative ionization mass spectrometry (REIMS).

### Embodiment 38

The method of any one of embodiments 23 to 37, wherein at least one of said plant and second plant is a transgenic plant.

### Embodiment 39

A device comprising

a means for in vivo rapid evaporating at least one metabolite of a plant or part thereof

ii) an analysis unit comprising means for determining the value of at least one parameter characteristic of said at least one metabolite

iii) an evaluation unit comprising a data storage unit and means for comparing the value of said at least one parameter to said reference amount.

### Embodiment 40

The device of embodiment 39, wherein said device is a mobile device.

### Embodiment 41

The device of embodiment 39 or 40, further comprising a plant or part thereof known to be in a specific metabolic state and/or a plant or part thereof known not to be in said metabolic state.

### Embodiment 42

Use of a device according to any one of embodiments 30 to 41 for determining a metabolic state of a plant, preferably according to the method of any one of embodiments 1 to 22; and/or for in vivo determining metabolite distribution in a plant or part thereof, preferably according to the method of any one of embodiments 23 to 38.

### Embodiment 43

A data collection, preferably a database, comprising reference values obtained from a plant known to be in specific metabolic state and/or from a plant known not to be in said metabolic state obtained by

a) rapid evaporating at least one metabolite of said plant or part thereof, and

b) determining the amount of at least one metabolite characteristic of said metabolic state.

### Embodiment 44

A data carrier comprising the database or the data of the database of embodiment 43.

Further optional features and embodiments of the invention will be disclosed in more detail in the subsequent description of preferred embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

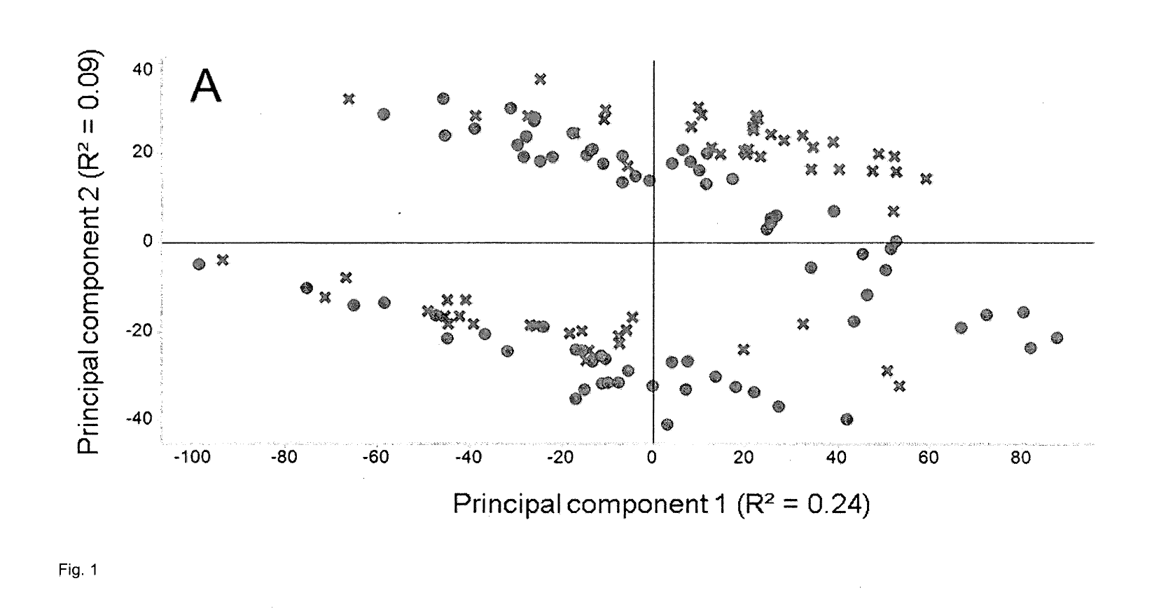

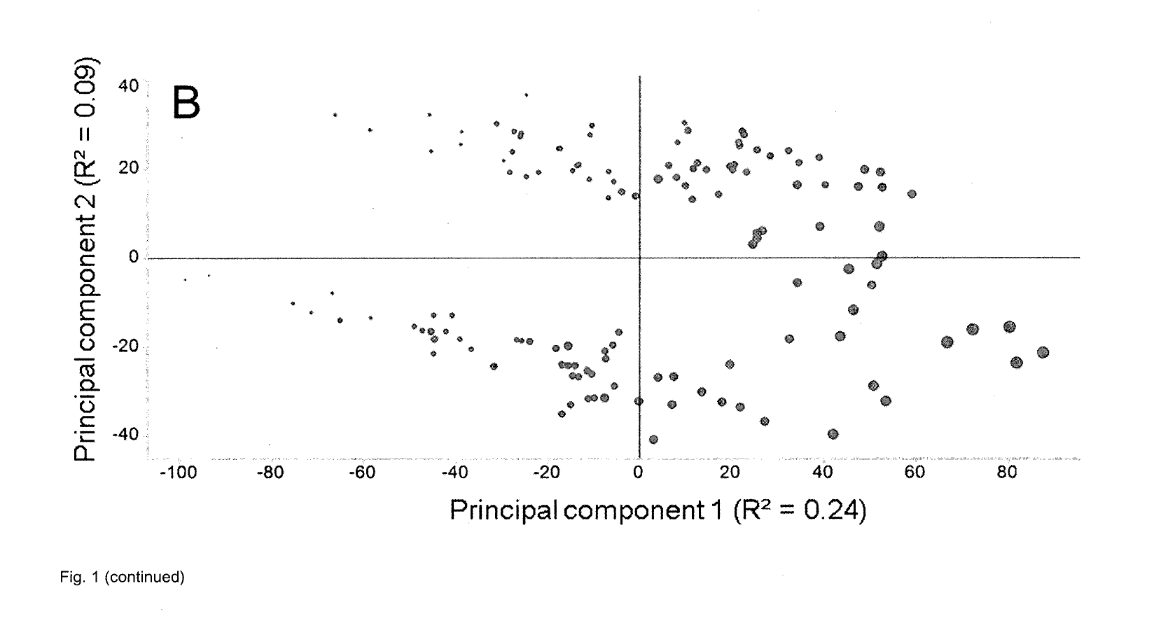

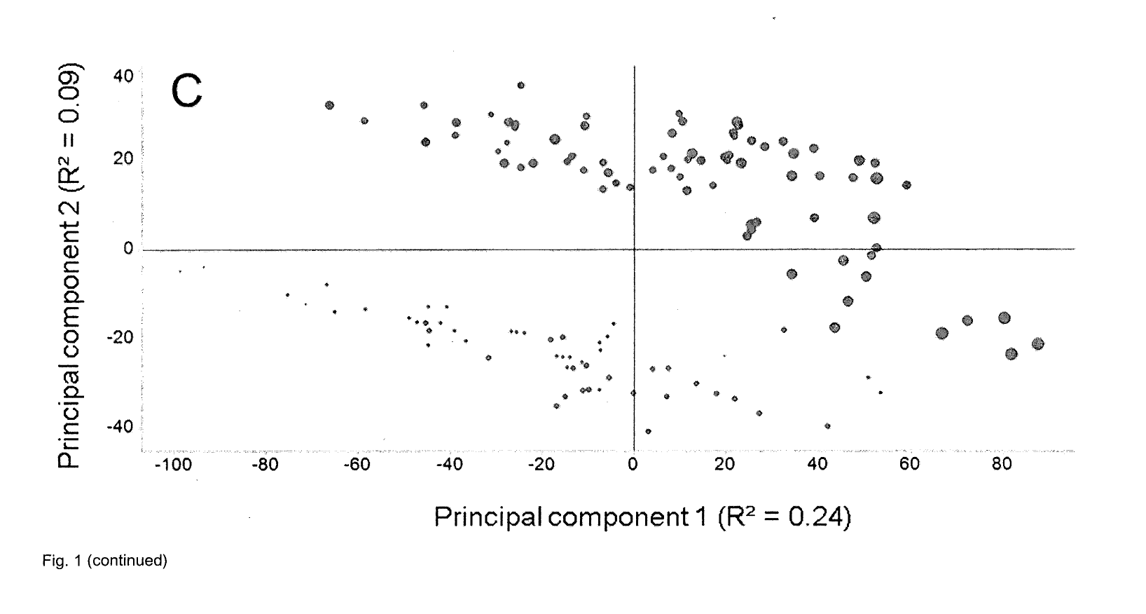

FIG. 1: PCA of data from corn leaves normalized to the sum of the intensities of all peaks in each MS sample as described in Example 5. A, Plot of score values from principal components 1 and 2, with circles representing individual MS samples from well-watered plants and crosses representing individual MS samples from drought-stressed plants. B, Plot of score values from principal components 1 and 2, with circle sizes indicating the weighted sum of peak intensities after normalization. C, Plot of score values from principal components 1 and 2, with circle sizes indicating the sum of peak intensities before normalization.

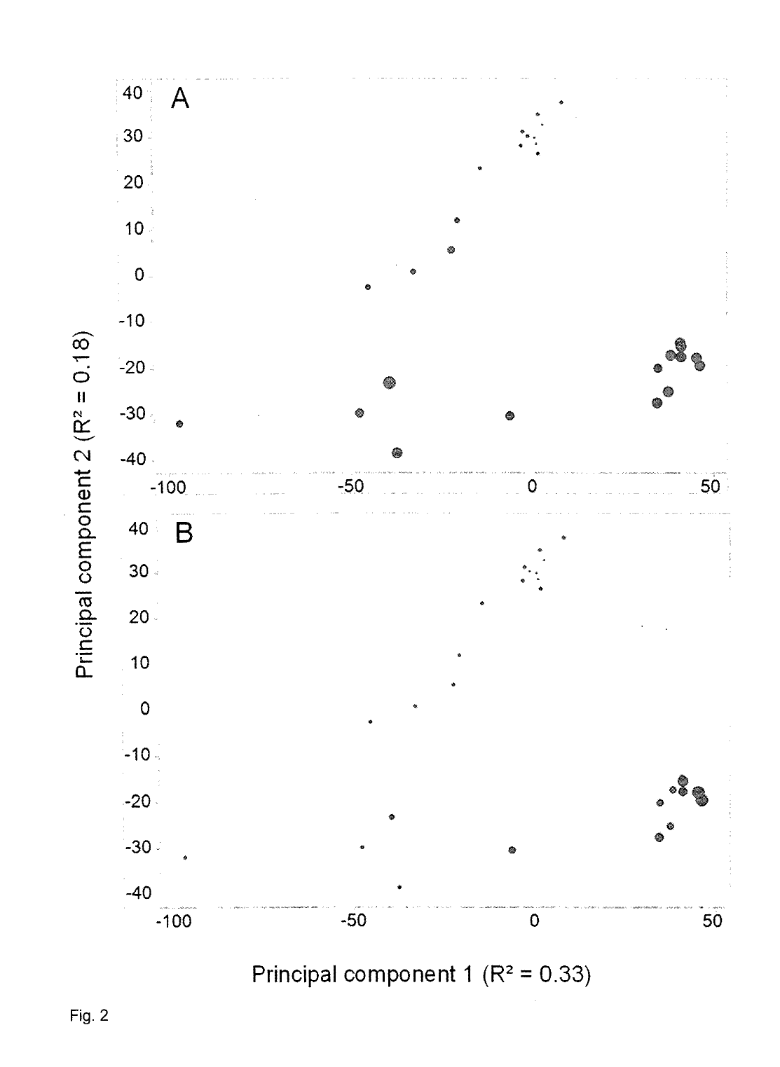

FIG. 2: PCA of data from chrysanthemum leaves normalized to the sum of the intensities of all peaks in each MS sample as described in Example 5. A, Plot of score values from principal components 1 and 2, with circle sizes indicating the weighted sum of peak intensities after normalization. B, Plot of score values from principal components 1 and 2, with circle sizes indicating the sum of peak intensities before normalization.

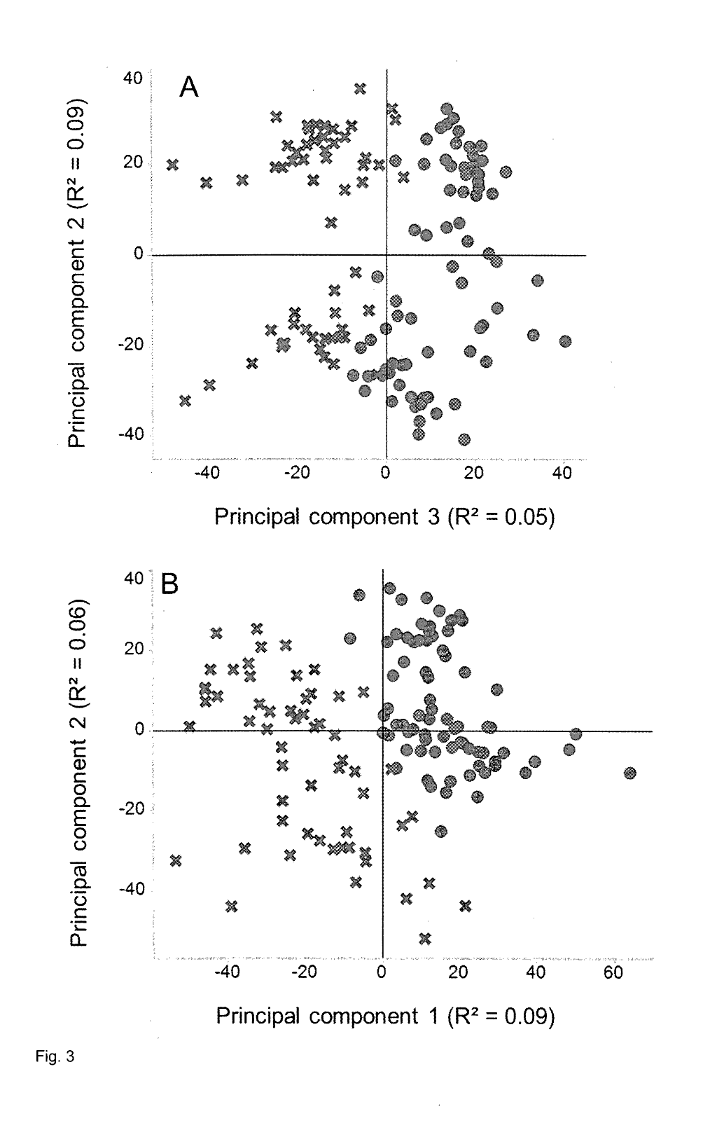

FIG. 3: PCA of normalized data from corn leaves before and after correction for the two peak sum parameters as confounders as described in Example 5. A, Plot of score values from principal components 2 and 3 before compensation. B, Plot of score values from principal components 1 and 2 after compensation. In both A and B, circles represent individual MS samples from well-watered plants and crosses represent individual MS samples from drought-stressed plants.

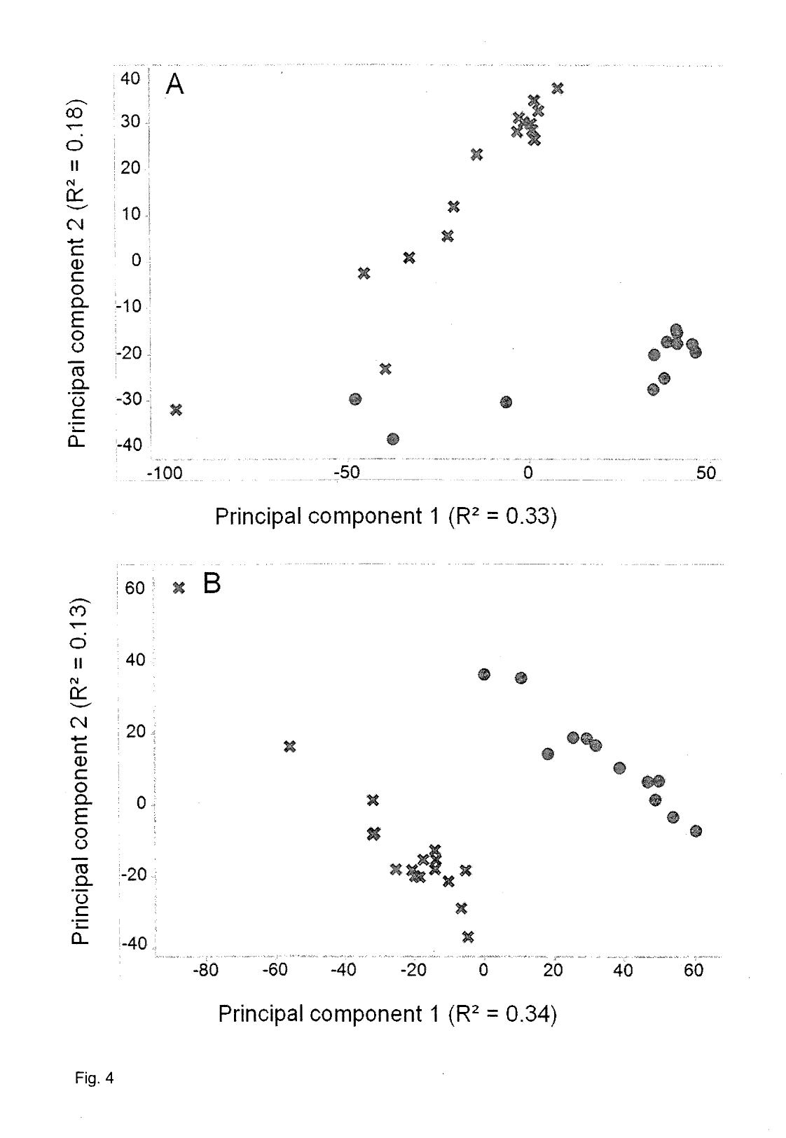

FIG. 4: PCA of normalized data from chrysanthemum leaves before and after correction for the two peak sum parameters as confounders as described in Example 5. A, Plot of score values from principal components 1 and 2 before compensation. B, Plot of score values from principal components 1 and 2 after compensation. In both A and B, circles represent individual MS samples from plants with red flowers and crosses represent individual MS samples from plants white flowers.

### EXAMPLES

### Example 1: Generation of a Reference Spectrum for Drought Stress

In this experiment, a plant screening for determining the metabolic state of a plant by identification of a stress pattern under drought stress is performed.

In a first step of the experiment, plants (transgenic or non-transgenic plants) are grown in potting soil under normal conditions until they approach the reproductive stage. A subgroup of the plants are then transferred to a “dry” section where irrigation is withheld. The other subgroup of plants are held under normal growing conditions without exposure to drought stress.

After exposure to drought stress, the treated plants as well as the control plants are subjected to rapid evaporative ionization coupled with mass spectrometry. Rapid evaporative ionization mass spectrometry enables the production, collection and transfer of tissues vapors to a mass spectrometer for subsequent detection.

The vapor for subsequent MS analysis is formed by using a commercially available electrosurgical devices which allows for rapid heating of the tissue. For the in vivo analysis, a plant tissue of interest is subjected to joule heating without detaching the tissue being analyzed from the plant body. Subsequently, as stated above the vapor harboring the ions is transferred to a mass spectrometer, the molecules are analyzed and spectral data recorded.

Thereafter, the spectral data of the control plants are compared to the spectral data of the plants treated with drought stress. Those spectra are statistically analyzed in order to identify significant differences between the two subgroups of plants that can be ascribed to the drought stress treatment. The information on the spectral differences and characterization according the applied stress is used to construct a reference spectral library. The library is used in a second step in which plants are classified according their spectral composition based on the predefined stress pattern.

### Example 2: Cultivation and Sampling of Corn Plants

Corn (Zea mays) plants were grown in a greenhouse without air conditioning and without artificial illumination over 6 weeks with regular watering twice a day. After week 6, the plants submitted to drought stress were not watered for three days and then only received limited amount of water (200-300 mL) per day for one further week. The control plants were grown in parallel and were watered twice a day as during the first six weeks of culture.

After 7.5 weeks, the leaves of the corn plants were cut, inserted in a falcon tubes and quenched in liquid nitrogen. The frozen samples, which are referred to as ‘plant samples’ elsewhere herein, were shipped on dry ice to the laboratory for analysis.

The vapor for subsequent MS analysis was formed by using a commercially available electrosurgical device (Radiosurg 2200, Meyer-Haake, Wehrheim, Germany), which allowed for rapid heating of the tissue as described by Balog et al., 2010 (cf. above). Each plant sample was measured five times in a sequence. One such measurement is referred to as ‘MS sample’ elsewhere herein.

### Example 3: Cultivation and Sampling of Chrysanthemum Plants

Chrysanthemums of red or white color were purchased from a local shop and kept in a winter garden for 48 hours before analysis.

For the in vivo analysis, a plant tissue of interest was sampled without detaching the tissue being analyzed from the plant body. This is referred to as ‘plant sample’ elsewhere herein. The chrysanthemum leaves were held between the two electrodes of a bipolar forceps device. The electrical current applied heated up the leaves and thus produced an aerosol, which was aspirated into the opening of the electrodes as described by Strittmatter et al., 2013 (Chemical Communications, 49:6188-6190).

Each plant sample was measured five times in a sequence. One such measurement is referred to as ‘MS sample’ elsewhere herein.

### Example 4: Mass Spectrometry and Signal Processing

The vapors created from the electrosurgical units were transferred to a Waters Xevo G2 XS Q-ToF using a polytetrafluoroethylene (PTFE) transfer line. Mass spectra were collected in negative mode in the mass range from 50 to 1200 Da. 16 mass spectra were recorded for each MS sample.

After collection of the mass spectra, the signals were further processed using Genedata software (Genedata AG, Basel, Switzerland). The intensities of the mass signals were averaged across all mass spectra recorded from the same MS sample. Then, the measured mass over charge ratios (m/z values) were adjusted in-silico using palmitic acid (m/z: 255.2331) for internal mass calibration, followed by a background subtraction. The mass signals were integrated in all MS spectra using a curvature-based peak detection method. The intensities of the integrated mass signals (referred to as ‘peaks’ elsewhere herein) were exported for data normalization and statistical analysis. Peaks originating from different MS samples, but having the same m/z value after in-silico correction, are referred to as ‘corresponding peaks’ elsewhere herein.

### Example 5: Data Normalization and Statistical Analysis

Data were normalized by the sum of the intensities of all peaks in each MS sample. Alternatively, data can be normalized by the median of the intensities of all peaks in each MS sample.

After normalization, the dataset was checked for the presence of outlier samples by a principal component analysis (PCA). The PCA was performed using the software package ‘ropls’ (Thevenot et al. (2015), Journal of Proteome Research 14:3322, R package version 1.4.6) for the software environment R. The input data were centered and scaled to unit variance. MS samples identified as outliers by visual inspection of plots showing score values were excluded from further data processing. The PCA was repeated on the dataset from which the outlier samples were excluded.

It was found that the distribution of the MS samples in plots showing score values correlated not only with factors describing the metabolic state of the plant or part of the plant which was sampled, but also with two confounding factors (FIGS. 1 and 2). One of said two confounding factors was the sum of the intensities of all peaks in the MS sample before normalization. The other one of said confounding factors was the weighted sum of the intensities of all peaks in each MS sample after normalization. The intensity of each peak in said weighted sum was weighted according to the relative standard deviation (RSD) of corresponding peaks across all MS samples. The RSD was calculated as the standard deviation of corresponding peaks across all MS samples, divided by the mean value of the same peaks. Missing values were ignored. This as well as similar ways to calculate the RSD are well known to the skilled person and are equally suited for this purpose. These two confounding factors are referred to collectively as ‘peak sum parameters’ elsewhere herein.

An analysis of variance (ANOVA) was conducted on the normalized dataset using a mixed effects model. The ANOVA model incorporated the two peak sum parameters described in the previous paragraph as fixed factors in addition to one or more fixed factors describing the metabolic state of the plant or part of the plant which was sampled. In addition, the ANOVA model contained a unique identifier of the plant sample as random factor to account for the fact that five MS samples were taken from each plant sample. The ANOVA was performed using the software package ‘nlme’ (Pinheiro et al. (2016), CRAN.R-project.org, R package version 3.1-89) for the software environment R.

In the case of the corn plants, the description of the ANOVA model in R syntax was:

Fixed part of the model: Peak intensity ˜sum of peak intensities before normalization+weighted sum of peak intensities after normalization+water availability

Random part of the model: Peak intensity ˜1|unique identifier of the plant sample

TABLE 1
Number of peaks which were significantly changed for each factor in the corn plants:

| | | Percentage of peaks | Probability that at least |
|---|---|---|---|
| | Number of peaks | which were | the same number |
| | which were | significantly changed, | of significant changes |
| | significantly changed | based on | would be observed |
| | in the ANOVA | a total number of 5881 | by chance, assuming |
| Factor | F-statistics (p < 0.05) | peaks per sample | a binomial distribution |
| Sum of peak | 4362 | 74% | <0.001 |
| intensities before | | | |
| normalization | | | |
| Weighted sum | 4223 | 71% | <0.001 |
| of peak intensities | | | |
| after | | | |
| normalization | | | |
| Water availability | 2156 | 36% | <0.001 |

Peaks which were significantly changed for factor ‘water availability’ can be used to determine the metabolic state of plants under drought stress and distinguish said metabolic state from that of well-watered control plants.

In the case of the chrysanthemum plants, the description of the ANOVA model in R syntax was:

Fixed part of the model: Peak intensity ˜sum of peak intensities before normalization+weighted sum of peak intensities after normalization+color

Random part of the model: Peak intensity ˜1|unique identifier of the plant sample

TABLE 2
Number of peaks which were significantly changed for each factor in the
chrysanthemum plants:

| | | Percentage of peaks | Probability that at least |
|---|---|---|---|
| | Number of peaks | which were | the same number |
| | which were | significantly changed, | of significant changes |
| | significantly changed | based on | would be observed |
| | in the ANOVA | a total number of 5382 | by chance, assuming |
| Factor | F-statistics (p < 0.05) | peaks per sample | a binomial distribution |
| Sum of peak | 1299 | 24% | <0.001 |
| intensities before | | | |
| normalization | | | |
| Weighted sum | 2211 | 41% | <0.001 |
| of peak intensities | | | |
| after | | | |
| normalization | | | |
| Color | 1624 | 30% | <0.001 |

Peaks which were significantly changed for factor ‘color’ can be used to determine the metabolic state of petals of a certain color and distinguish said metabolic state from that of petals of another color.

Alternatively, other ANOVA models can be used for the analysis of the data. How to devise such ANOVA models is well known to the skilled person. The skilled person can apply, for example, fixed-effect models, mixed-effect models, or hierarchical models. The fact that alternative ANOVA models can be devised does not limit the general applicability of the illustrated approach.

The normalized dataset was corrected for the variation which correlated with the two peak sum parameters by subtracting from the normalized dataset the predicted effects attributable to these two confounding factors. This confounder correction was performed using the same ANOVA model which was used further above in this Example to investigate the influence of experimental factors on the metabolic state of the plant.

Alternatively, other ANOVA models can be used to compensate the normalized dataset for the variation which correlates with the two peak sum parameters. For example, in applications where information on factors describing the metabolic state of the plant or other information on the experimental design is lacking, an ANOVA model can be applied where the fixed part incorporates only the two peak sum parameters. How to devise such ANOVA models is well known to the skilled person. The skilled person can apply, for example, fixed-effect models, mixed-effect models or hierarchical models. The fact that alternative ANOVA models can be devised does not limit the general applicability of the illustrated approach.

The normalized and confounder-corrected dataset was used as input for a PCA, which was performed using the software package ‘ropls’ (Thevenot et al. (2015), Journal of Proteome Research 14:3322, R package version 1.4.6) for the software environment R. The input data were centered and scaled to unit variance.

FIG. 3 shows that after correction for the two peak sum parameters as confounders, the metabolic state of corn plants under drought stress can be distinguished from the metabolic state of well-watered control plants in the first principal component (Panel B), while before confounder correction, the metabolic state of corn plants under drought stress can be distinguished from the metabolic state of well-watered control plants only in the third principal component (Panel A).

FIG. 4 shows that after correction for the two peak sum parameters as confounders, the metabolic state of flowers of red-flowering chrysanthemum plants can be clearly distinguished from the metabolic state of flowers of white-flowering chrysanthemum plants in the first principal component (Panel B). Before confounder correction, the distinction between flowers form red-flowering and white-flowering plants was less clear (Panel A).

Alternatively, other multivariate approaches can be used to classify the metabolic state of the plant. Such approaches are, for example, logistic regression as an example of linear classification algorithms, support vector machines as an example of machine learning approaches, or random forests as an example of decision tree-based approaches. How to apply such approaches is well-known to the skilled person.

1. A method for determining a metabolic state of a plant or part thereof comprising
a) rapid evaporating a multitude of metabolites of said plant or part thereof;
b) determining the amount of at least one metabolite characteristic of said metabolic state; and
c) thereby, determining a metabolic state of a plant or part thereof.

2. The method of claim 1, wherein rapid evaporation is induced by applying high-frequency alternating current to said plant or part thereof.

3. The method of claim 1, wherein said high-frequency alternating current is applied to said plant or part thereof by means of two electrodes of about equal size.

4. The method of claim 1, wherein the high-frequency alternating current is applied to said plant or part thereof by means of electrosurgical equipment.

5. The method of claim 1, wherein said steps a) and b) are performed by rapid evaporative ionization mass spectrometry (REIMS).

6. The method of claim 1, wherein the part of the plant on which rapid evaporation is performed is not removed from the plant.

7. The method of claim 1, wherein said rapid evaporating is performed on a small area on the intact plant or plant part.

8. The method of claim 1, wherein said metabolic state is a biotic stress metabolic state or an abiotic stress metabolic state.

9. The method of claim 1, further comprising the step of b1) comparing the amount of said at least metabolite to an amount of the same metabolite determined in a plant or part thereof known to be in said metabolic state and/or determined in a plant or part thereof known not to be in said metabolic state.

10. A method for in vivo determining metabolite distribution in a plant or part thereof comprising
a) in vivo rapid evaporating at least one metabolite of interest in at least a first and a second location of said plant or part thereof
b) determining the amounts of at least one metabolite at said first and a second location, and,
c) thereby, in vivo determining metabolite distribution in a plant or part thereof.

11. The method of claim 10, wherein rapid evaporation is induced by applying high-frequency alternating current to said plant or part thereof.

12. The method of claim 10, wherein said high-frequency alternating current is applied to said plant or part thereof by means of two electrodes of about equal size.

13. The method of claim 10, wherein the high-frequency alternating current is applied to said plant or part thereof by means of electrosurgical equipment.

14. The method of claim 10, wherein said steps a) and b) are performed by rapid evaporative ionization mass spectrometry (REIMS).

15. The method of claim 10, further comprising the step of comparing the metabolite distribution determined in step c) to the metabolite distribution in a second plant.

16. A device comprising
i) a means for in vivo rapid evaporating at least one metabolite of a plant or part thereof
ii) an analysis unit comprising means for determining the value of at least one parameter characteristic of said at least one metabolite
iii) an evaluation unit comprising a data storage unit and means for comparing the value of said at least one parameter to said reference amount.

17. (canceled)

18. A data carrier comprising a data collection, comprising reference values obtained from a plant known to be in specific metabolic state and/or from a plant known not to be in said metabolic state obtained by
a) rapid evaporating at least one metabolite of said plant or part thereof, and
b) determining the amount of at least one metabolite characteristic of said metabolic state.

19. The device according to claim 16, wherein rapid evaporation is or was induced by applying high-frequency alternating current to said plant or part thereof.
